(19) United States
(12) Patent Application Publication
(10) Pub. No.: US 2002/0106718 A1
(43) Pub. Date: Aug. 8, 2002

(54) Cell transfer device

(75) Inventors: Peter P. Gombrich, Chicago, IL (US); Richard A. Domanik, Libertyville, IL (US)

Correspondence Address: MERCHANT & GOULD PC, P.O. BOX 2903, MINNEAPOLIS, MN, 55402-0903, US

(73) Assignee: MOLECULAR DIAGNOSTICS, INC., Chicago, IL

(21) Appl. No.: 10/004,704

(22) Filed: Dec. 4, 2001

Related U.S. Application Data
(60) Provisional application No. 60/251,143, filed on Dec. 4, 2000.
(60) Provisional application No. 60/277,759, filed on Mar. 21, 2001.
(60) Provisional application No. 60/280,208, filed on Mar. 30, 2001.
(60) Provisional application No. 60/322,009, filed on Sep. 13, 2001.

(51) Int. Cl. G01N 1/30; G01N 33/48; C12M 1/00

(52) U.S. Cl. 435/40.5; 435/283.1

(57) ABSTRACT

Cellular samples such as cervical cells can be obtained from a cell suspension and then transferred to a microscope slide for analysis. The cells can be retrieved from the cell suspension using an inexpensive, easy to use device that requires no instruments or ancillary devices, minimal operator skill and training, and is potentially sufficiently low cost that it is suitable for use in mass screening programs.

### RELATED APPLICATIONS

This application claims benefit of U.S. Provisional Applications Serial No. 60/251,143, filed Dec. 4, 2000 entitled “TRIAGE DEVICE FOR CAPTURING CELLULAR MATERIAL”; Ser. No. 60/277,759, filed Mar. 21, 2001 entitled “METHOD AND APPARATUS FOR TRANSFERRING CELLULAR SAMPLES TO A MICROSCOPE SLIDE”; Ser. No. 60/280,208, filed Mar. 30, 2001 entitled “SLIDE PREPARATION DEVICE”; and Ser. No. 60/322,009, filed Sep. 13, 2001 entitled “VOLUMETRIC SLIDE PREPARATION DEVICE”. Each application is specifically incorporated in its entirety by reference herein.

### TECHNICAL FIELD

The invention relates generally to screening methods and more specifically to means and methods of capturing, transferring and analyzing cells. More specifically, the invention relates to means and methods of transferring cells to a microscope slide.

### BACKGROUND

Many forms of cancer can be successfully controlled or treated if the condition is detected sufficiently early in the development of the cancer. As a result, a number of screening tests and investigative methods have been developed. These include cytological procedures such as the PAP test and imaging modalities such as X-ray and ultrasound. However, optimal detection requires morphological examination of the tissue in question.

One example of a cancer with substantial cure rates if detected early enough is cervical cancer, which frequently begins as a precancerous lesion of the cervix. These lesions are also known as cervical intraepithelial neoplasia. If left untreated, these lesions can deepen over time and ultimately develop into an invasive cancer of the cervix and associated tissues. Fortunately, early detection followed by appropriate treatment results in a very high cure rate for cervical cancer.

Therefore, it is beneficial for at least certain factions of the female population to undergo regular screening. These factions include patients with previous cervical abnormalities and those who have a family history of cervical abnormalities. Women who are sexually active are at greater risk and should undergo regular screening, as are those who test positive for HPV (human papillomavirus). This is a sexually transmitted virus that in some forms can cause genital warts.

During the 1940's, Dr. George Papanicolaou developed a screening test which bears his name and which has become the most widely used screening technique for detecting abnormal cervical cells. Today, this test is known more commonly as the PAP test or the PAP smear test. The PAP test is typically performed in the physician's office as part of a routine gynecological examination.

The Pap test involves collecting exfoliated cells from the cervical epithelium using a brush, spatula, swab or similar device and smearing the collected cells onto a microscope slide. This frequently results in slides that are less than ideal for examination and evaluation purposes. In particular, the collected cells can be deposited on the slide in thick heaps and clumps that obscure underlying cells are obscured. In addition to the desired cervical epithelial cells, blood and mucus are frequently collected by the cell collection device. When transferred to a microscope slide via a smearing process, this blood and mucus can obscure the desired cells.

The “liquid-based preparation” (LBP) is a method that was specifically developed to address these limitations. In the LBP procedure, the cells on the collection device are released into a liquid medium to form a cell suspension. The cell release and suspension process can be sufficiently vigorous to disperse clumps and clusters of cells. Furthermore, the fluid into which the cells are released often contains mucolytic agents and agents that selectively lyse red blood cells. Thus, the resulting cell suspension can be relatively homogeneous and relatively free of interfering agents. Some LBP procedures incorporate additional means for further perfecting the cell suspension.

Once prepared, cells from this suspension can be transferred to a microscope slide by a variety of methods. One option, as exemplified by the “AutoCyte” process commercialized by Tri-Path Imaging Corporation, is to allow the cells in the suspension to settle onto the microscope slide under the influence of gravity. A similar technique, such as is exemplified by the “CytoSpin” method commercialized by Thermo-Shandon causes the cells to settle onto the slide under the influence of an augmented gravitational field provided by centrifugation. Another common technique is to collect cells from the cell suspension onto the surface of a membrane filter via a filtration process and then transfer these collected cells to the slide by bringing the cell-coated filter into contact with the slide. The Cytyc procedure commercialized by Cytyc Corporation is an example of this type of procedure.

These methods rely on interfacial forces to adhere the cells to the slide. In some cases such as the CytoSpin and Cytyc processes where externally applied physical forces bring the cells into intimate physical contact with the slide, direct interactions between the surfaces of the cells and the slide are sufficient to cause the cells to be retained on the slide. In other cases, it is desirable to augment cell retention by applying an adhesion promoter such as poly-1-lysine or an aminosilane to the slide before cell deposition. The intent in any of these cases is to deposit a nominal monolayer of cells onto the slide surface.

The number of cells collected using any of the commonly employed cell collection devices is highly variable and as a consequence, the concentration of cells in the resulting LBP suspension is similarly variable. However, the number of cells that can be accommodated as a monolayer over a predefined area is limited by geometric factors. In clinical practice, the number of cells in the suspension usually far exceeds the number of cells required to form the desired monolayer. This means that the cells forming the monolayer will constitute a sub-sample of the cells that were present in the original cell suspension.

One way of obtaining the required sub-sample is to estimate or determine the concentration of cells in the original suspension; calculate the volume of suspension that contains the number of cells required to form the desired monolayer; obtain an aliquot of the cell suspension that is of the appropriate volume to contain the desired number of cells; and use this aliquot of cell suspension in the preparation of the slide. Cell concentration can be determined or estimated by any of a variety of methods ranging from direct cell counts using a hemocytometer to turbidimetric measurements to visual estimation. Aliquoting the desired volume of cell suspension can be performed via some form of volumetric pipetting procedure.

Another common method of obtaining the desired sub-sample makes use of the characteristics of a membrane filter of the “track etch” type such as the “Nucleopore” filters manufactured by Millipore Corporation. These filters consist of a thin homogeneous membrane that is penetrated by a defined density of approximately cylindrical pores that have defined characteristics and which are oriented approximately perpendicular to the membrane surface. Track etch filters having pore sizes in the 5 to 10 micron diameter range are commonly employed.

When a cell suspension is passed through such a filter, the cells, which are too large to pass through the pores of the filter, are collected on the surface of the filter while the fluid passes through. Fluid flow through the filter, however, ceases when all of the pores in the filter become plugged or covered by collected cells. Since the pore density and the size of the membrane are known and controlled, the number of cells that can be captured on a track etch filter is likewise controlled. The cells that are captured on the filter represent a sub-sample of the cells present in the original suspension and can either be directly transferred to the slide via a contact process or can be released into a known volume of cell-free fluid to form a suspension of known cell concentration.

As is intimated by the foregoing, the preparation of a cytological microscope slide using any of the current LBP methods either requires a skilled technician to perform multiple manual steps or the use of complex and expensive instrumentation to perform the same operations. While the use of these alternatives can be economically justified in some low volume and high volume clinical laboratory environments, respectively, they are inappropriate in many situations. For example, in some countries, the examining physician not only collects the cell sample, but prepares and examines the resulting slide. At the opposite extreme, in many public health mass screening programs it is desirable to screen large numbers of patients at minimum cost using relatively unskilled labor sufficiently rapidly that slide preparation and evaluation can be completed before the patient leaves the screening area. In other words, current LBP slide preparation methods are inappropriate in cases where they must be performed outside of a classical laboratory environment.

All of these methods use disposable devices that are too expensive for use in mass screening programs. A need remains for an inexpensive and simple method and means for retrieving cells from a cellular sample and preparing a suitable sample for examination under a microscope. A need remains for means and methods for the preparation of microscope slide specimens for cytological analysis that can be performed at low cost by relatively unskilled labor outside of a classical laboratory environment.

### SUMMARY

The present invention is directed to means and methods for the preparation of microscope slide specimens for cytological analysis that can be performed easily and inexpensively. In particular, cellular samples such as cervical cells can be obtained from a cell suspension and then transferred to a microscope slide for analysis. The cells can be retrieved from the cell suspension using an inexpensive, easy to use device that requires no instruments or ancillary devices, minimal operator skill and training, and is potentially sufficiently low cost that it is suitable for use in mass screening programs.

Accordingly, an embodiment of the present invention is found in a device for transferring cells from a cell suspension onto a substrate. The device includes segregation means to segregate the cells in a portion of the cell suspension from the cell suspension and transfer means to transfer the segregated cells to a solid surface. The segregated cells are deposited on the solid surface in an approximation of a monolayer and the remainder of the cell suspension is recoverable for other use.

The invention is also found in a device for depositing cells from a fluid suspension onto a solid substrate. The device includes a first chamber for containing the fluid suspension, the chamber being divided into two or more contiguous zones. The device also includes a second chamber for receiving excess fluid suspension and a channel through which excess fluid suspension can be displaced from the first chamber to the second. Retaining means of retaining the solid substrate relative to the first chamber are also included. The device also includes a displacement device that has a member slideable within the first chamber. The displacement device includes at least a body element, a porous element, and a fluid absorbing element.

The invention is also found in a method for depositing cells from a fluid suspension onto a solid substrate. A slideable element is moved to trap a predetermined sub-sample of the fluid suspension in a chamber and is moved further to transfer cells in the fluid suspension to a porous element that is attached to the slideable element with simultaneous absorption of cell-free fluid by an absorbing element. The cells are transferred from the porous element to the solid support by pressure contact.

The invention is also found in a device for retrieving cells from a cell collection device. The device includes a housing. Within the housing are a first chamber, a second chamber and a third chamber. The second chamber is configured to accept a cervical spatula. The first, second and third chambers are each in fluid communication with each other.

Other features and advantages of the present invention will be apparent from the following detailed description and drawings.

### BRIEF DESCRIPTION OF THE FIGURES

FIG. 1 is a diagrammatic cross-sectional view of a slide preparation device in accordance with an embodiment of the present invention.

FIG. 2 is a diagrammatic cross-sectional view of a slide preparation device in accordance with another embodiment of the present invention.

FIG. 3 is a diagrammatic cross-sectional view of a slide preparation device in accordance with yet another embodiment of the present invention.

FIG. 4 is a top plan view of a one-way valve as used in the slide preparation device of FIGS. 2 and 3.

FIG. 5 is a schematic illustration of a cell transfer device according to an embodiment of the present invention, shown in a raised position.

FIG. 6 is a schematic illustration of the cell transfer device of FIG. 5, shown in a lowered position.

FIG. 7 is an exploded perspective view of a clamshell device in accordance with an embodiment of the present invention.

FIG. 8 is a perspective view of the clamshell device of FIG. 7, illustrating placement of a spatula within the device. In this Figure, the cover and the membrane support have both been removed from the device.

FIG. 9 is a perspective view of the clamshell device of FIG. 7, shown in a cell washing configuration. The cover is in place over the spatula.

FIG. 10 is a perspective view of the clamshell device of FIG. 7, shown in a cell transfer configuration. The membrane support (and membrane) are positioned to accept cells from the cell collection fluid within the device.

FIG. 11 is a perspective view of the clamshell device of FIG. 7, shown without the spatula but with the membrane support in position.

### DETAILED DESCRIPTION

Cellular samples such as cervical cells can be obtained from a cell suspension and then transferred to a microscope slide for analysis. The cells can be retrieved from the cell suspension using an inexpensive, easy to use device that requires no instruments or ancillary devices, minimal operator skill and training, and is potentially sufficiently low cost that it is suitable for use in mass screening programs. Cells can be transferred using devices that employ gravity settling or by devices that employ pressurization.

The invention is directed to means and methods of capturing cellular samples such as cervical cells from a sampling device and transferring the cells to a membrane or microscope slide for analysis.

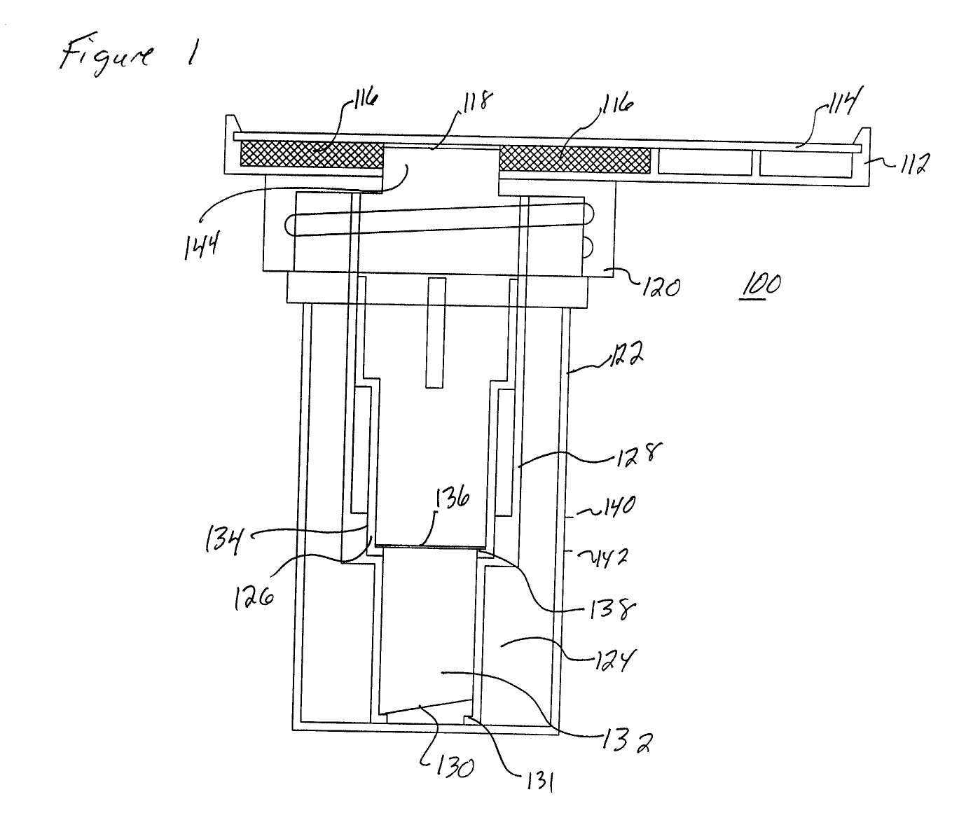

One particular device employs gravity settling and is illustrated for example in FIG. 1, which shows a gravitational settling device 100 that includes a vial 122 having a cap 120 that can be configured to incorporate a holder 112 for a microscope slide 114. The cap 120 can optionally include one or more fluid absorbers 116 and/or may incorporate a mesh screen 118 positioned adjacent to, but not in contact with the microscope slide 114. This device further incorporates a dip-type volumetric sampling structure that includes a body 126, an inlet valve 130 and an outlet valve 136 that can be attached to the cap 120 and the microscope slide holder 112 in either a fixed or sliding arrangement.

The fluid absorbers 116 can be made from any suitable material, providing of course that the material used has a sufficient fluid absorbency capacity. The fluid absorbers 116 can be sized and arranged to be able to capture substantially all of the fluid that impinges on the slide 114.

In a first embodiment, as illustrated in FIG. 1, there is attached to the cap 120 a sampling probe 124 that includes an inner element 126 and an outer element 128. The inner element 126 can be slidably positioned within the outer element 128. A lower portion of the outer element 128 forms a sampling chamber 132. In this, upper and lower are not limiting, but merely refer to the illustrated orientation of the sampling device 110. The sampling chamber 132 is defined in part by a one-way valve 130 that is positioned within the outer element 128 at the lower end of the sampling chamber 132 and by a vent 136 that is positioned (at the lower end of the inner element 126) at the upper end of the sampling chamber 132. In a preferred embodiment, the vent 136 is a high porosity hydrophobic material that permits air to pass through but resists the flow of water.

The inner element 126 also includes slots 134 that allow fluid to flow from the sampling chamber 132 towards the cap 120 when the sampling device 110 is inverted, as will be described in greater detail hereinafter.

In accordance with an embodiment of the invention as illustrated in FIG. 1, sub-sampling of the suspension is accomplished via the sampling probe 124. Prior to use, the lower end of the inner element 126, which is closed by a high porosity hydrophobic vent 136 (passes air, but not fluid), is seated against a feature 138 in the outer element 128 thus dividing the outer element into two compartments including a sampling chamber 132.

When the sampling probe 124 is immersed in the cell suspension, the suspension enters the sampling chamber 132 through the one-way valve 130 while the displaced air exists through the hydrophobic vent 136. Fluid entry into the sampling compartment 132 ceases when the advancing fluid front reaches the hydrophobic vent 136.

Transfer of the cells from the sampling chamber 132 to the microscope slide 114 is accomplished by inverting the sampling device 110, as illustrated in FIG. 1. This causes the inner slideable element 126 of the sampling probe 124 to be displaced toward the microscope slide 114 thus opening ports 134 that allow the sub-sample of the cell suspension to flow onto the microscope slide 114 through an aperture 144 that defines the size and shape of the area over which the cells are to be deposited. The cells are then allowed to settle from the suspension onto the slide 114 under the influence of gravity.

The vial 122 is selected, sized and contains a volume of cell preservative solution in accordance with the characteristics of the cell collection device(s) to be used in conjunction with the present invention. By way of example, the standard vial used in the Cytyc LBP slide preparation process has approximate dimensions of 34 mm (diameter) by 70 mm (height) with a nominal fluid capacity of about 50 ml and a nominal fluid contents of20 ml of preservative liquid.

The vial 122 can contain about 10 to 20 milliliters of fluid. As discussed previously, in preparing a microscope slide 114 it is preferable that only a particular fraction of the original sample volume be used. In a preferred embodiment, the sampling device 110 is configured to yield a 10 to 1 volumetric reduction. Thus, for an original sample volume of 20 milliliters, a volume of 2 milliliters will be captured for providing a sample on the microscope slide 114.

The dimensions and capacities of the vials used in other similar commercially available LBP processes vary considerably from the Cytyc example, but in all cases the inner dimensions of the vial and the fluid fill level in the vial are such that the cell containing portion of the cell collection device can be completely immersed and agitated in the preservative solution for the purposes of preparing the cell suspension. The cell preservative solutions used in the preparation of the cell suspensions for the LBP process generally incorporate a significant concentration of one or more lower alcohols such as ethanol. Thus all materials used in the construction of the present invention are selected to withstand immersion in such liquids.

The volumetric sampling structure in the present invention is sized on one hand in accordance with the dimensions of the area on the microscope slide 114 over which the cellular monolayer is to be deposited and, on the other hand, the anticipated fluid depth and cell concentration of the cell suspension in vial 122. If, by way of example, the maximum nominal number of cells required to form a monolayer on the microscope slide is determined to be 50,000 and the vial contains 20 ml of cell suspension at a concentration of between 10,000 and 30,000 cells per ml with the preponderant concentration being in the 20,000 to 25,000 cells per ml range, the volume of cell suspension to be taken from the vial in order to prepare a cellular monolayer from the majority of the samples encountered should be approximately 2 ml. This volume is defined by the dimensions of the sampling chamber 132. The height of chamber 132 is preferably less than the depth of the cell suspension in vial 122 to ensure proper filling of the chamber.

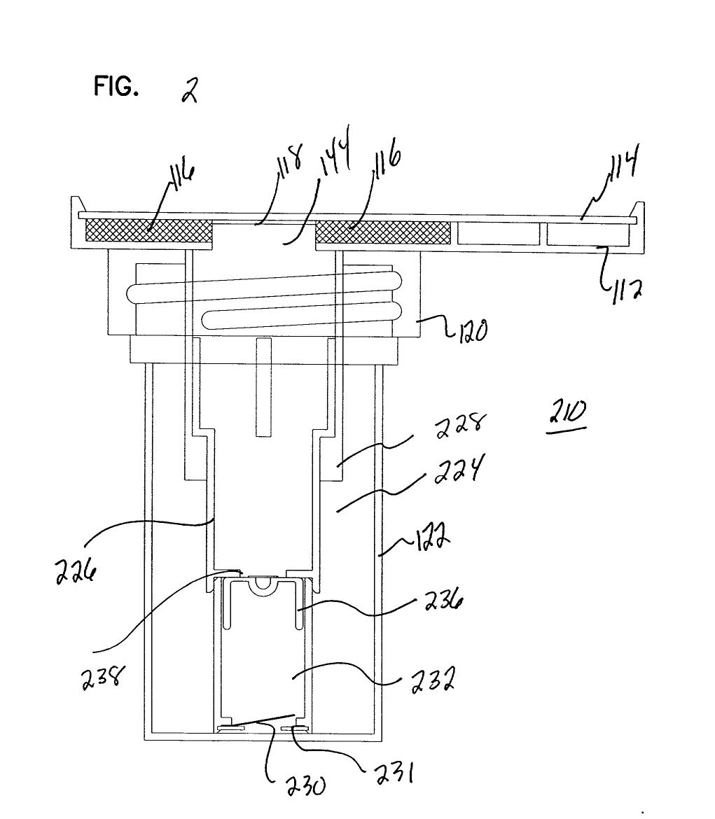

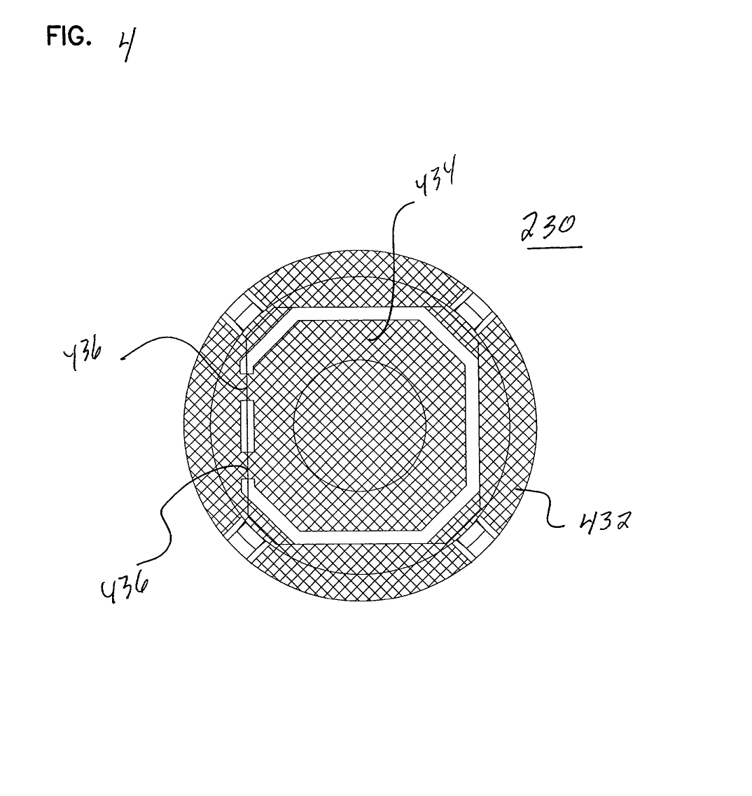

FIG. 2 illustrates another embodiment of the present invention. FIG. 2 shows a cap 120 to which is attached a sampling probe 224. The sampling probe 224 includes an inner element 226 and an outer element 228. The sampling probe 224 also includes a sampling chamber 232 that is separated from the inner element 226 by a float 236. The lower end of the sampling chamber 232 is defined in part by a one-way valve 230, which is illustrated in greater detail in FIG. 4.

The one-way valve 230 includes an outer portion 432 that is sized and configured to fit within the lower end of the sampling chamber 232. The one-way valve 230 includes a center portion 434 that is impervious to fluid flow and that is hingedly attached to the outer portion 432 through hinges 436. When the one-way valve 230 is in a closed position (not illustrated), the outer portion 432 meets with element 231 (131 in FIG. 1) to prevent the center portion 434 from swinging outwardly and allowing fluid to drain from the sampling chamber 232.

As illustrated for example in FIG. 2, the sampling probe 224 can be immersed in a cell suspension that has been placed within the vial 122. The cell suspension enters the sampling chamber 232 through the one-way valve 230 while the displaced air exits the sampling chamber 232 by traveling around and beyond the float 232. When the sampling chamber 232 is full of cell suspension, the float will contact the bottom end of the inner element 226 and thus will prevent any fluid from passing though aperture 238.

Slide preparation is initiated by inserting the cap 120 with the attached slide holder 112, the microscope slide 114 and the volumetric sampling device 226 into the vial 122 containing the previously prepared cell suspension. In its initial state before contact with the cell suspension, the inlet valve 230, which is shown schematically in FIG. 1 as a flapper valve, is in its closed position and the outlet valve 236, which is shown schematically as a float valve, is in its open position.

As the inlet end of the volumetric sampling device enters the cell suspension, hydraulic forces cause the inlet valve 230 to open thus admitting cell suspension into the sampling chamber 232. As the cell suspension enters the sampling chamber 232, bouyant forces cause the float valve 236 to rise with the fluid front until it contacts and forms a fluid tight seal against its seat 238. Closure of the outlet valve 236 causes entry of the cell suspension into the sampling chamber 232 to stop and due to the equalization of hydraulic forces, allows the inlet valve 230 to close. At this point, the sampling chamber 232 contains the desired volume of cell suspension in a manner that is isolated from the bulk cell suspension remaining in the vial 122.

Deposition of the monolayer is initiated by inverting the entire device. When inverted, bouyant forces cause the outlet valve 236 to open thus allowing the captured sub-sample of the cell suspension to drain through the body of the sampling device 226 onto the microscope slide 114. The pressure differential created by this action causes the inlet valve 230 to open to allow the sampling chamber to vent.

It is desirable, but not required for a mesh screen 118 to be placed adjacent to, but not in contact with the microscope slide 114 to prevent non-disaggregated clumps of cells from reaching the slide 114. The characteristics of this mesh screen are determined by the effectiveness of the cell dispersion method used during the preparation of the cell suspension. A nylon mesh screen having a nominal pore size of 500 microns is generally effective, but these characteristics can be adjusted to suit the requirements of the particular cell samples and suspension preparation method.

The cell suspension that has optionally been filtered through mesh screen 118 to remove cell aggregates is allowed to settle under the influence of gravity for a period of time that is determined by the height of the solution column above the microscope slide 114 and the density of the cell preservative solution. The height of the solution column is determined by the area over which cells are to be deposited and by the volume of cell suspension delivered to the slide by the sampling compartment 232. The settling time generally ranges from about three minutes for a short solution column and a low density preservative to about 15 minutes for a tall solution column and a high density preservative.

The cells that settle into contact with the surface of the microscope slide 114 bind to the surface of the slide through a combination of electrostatic and hydrophobic forces. Cells that settle into contact with other cells that are in turn bound to the surface of the microscope slide are held in place by weaker cell to cell adhesive forces. After settling has been completed, gentle swirling or agitation of the inverted device dislodges cells that are held to the microscope slide by cell to cell adhesion but does not dislodge those cells that are held in place by the stronger cell to microscope slide adhesive forces. The net result of settling followed by agitation is the formation of a nominal monolayer of cells attached to the surface of the microscope slide.

Depending upon the specifics of a particular embodiment for a particular application, it maybe desirable to interpose an adhesion promoting coating or layer between the surface of the microscope slide and the deposited cells. The use of materials such as poly-L-lysine and various aminosilanes for this purpose is well known in the art. Interposing such an adhesion promoter enhances the differential between the cell to slide and cell to cell binding forces and facilitates the perfection of the desired monolayer.

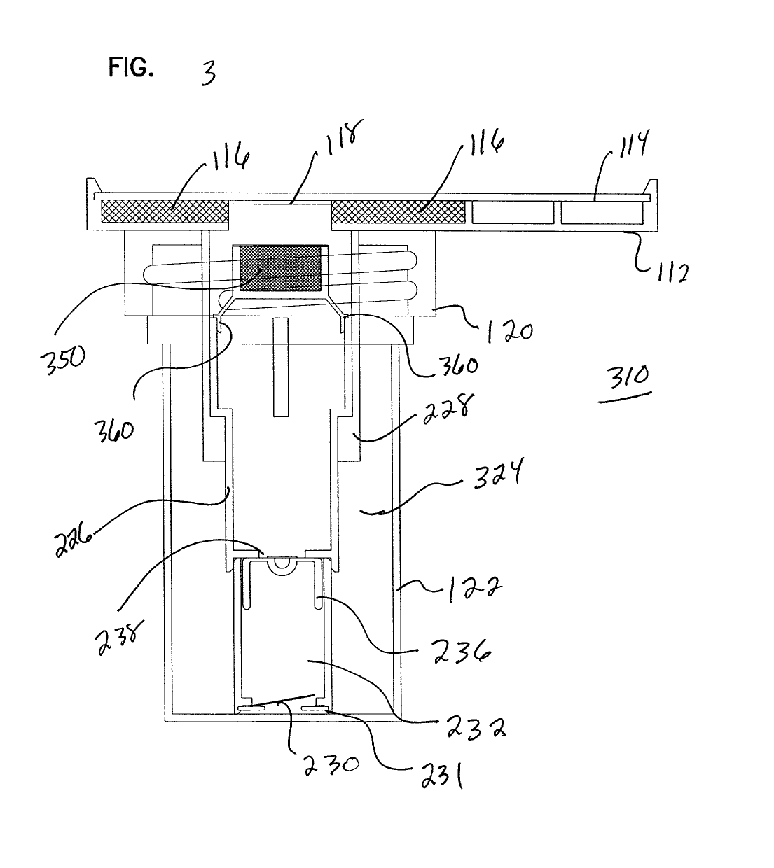

In another embodiment, as illustrated in FIG. 3, the outer element 228 can include a tamping device 350, which is attached to the outer element 228 at connection points 360. When the sampling device 310 is inverted, the tamping device 350 will move towards the slide 114 and will in effect press the cells onto the slide 114.

As illustrated in FIG. 1, an optional fluid absorber 116 may be juxtaposed to the cell deposition area on the microscope slide 114. At one extreme, which is preferred when, over the range of samples to be processed, the number of cells contained in the sampling chamber can be reasonably expected to consistently be less than the number required to form a confluent monolayer on the microscope slide, the dimensions and absorption characteristics of the absorber can be selected to absorb essentially all of the fluid delivered to the slide at a rate where the settling of cells onto the slide is completed before all of the fluid is absorbed. This offers the benefit that the microscope slide is nominally free of bulk fluid at the conclusion of the deposition process.

A second benefit is that this permits an “end of process” indicator to be integrated into the invention. In one embodiment, a small region (not shown) on the slide holder 112 is frosted. When the solution front in the absorber reaches the frosted area, its visual appearance changes from frosted to transparent and, if properly placed, can signal that the deposition and fluid capture processes are essentially completed. In another embodiment, a band of colored material (not shown) can be incorporated into the absorber 116. This colored material is selected such that it moves chromatographically with the fluid front in the absorber. Deposition can be considered to be complete when this colored material has migrated to a previously determined position. This disadvantages of absorbing all of the fluid are the potential for cell loss onto the absorber, a potential decrease in the uniformity of cell deposition due to fluid flow parallel to the surface of the microscope slide; and the potential for the deposition of localized multiple layers of cells.

At the opposite extreme, which is preferred when it cannot be predicted whether or not the number of cells contained in the sampling chamber will exceed the number required to form a monolayer, the absorber can be selected an configured to slowly absorb only a portion of the fluid. The advantage of this approach is that the height of the fluid column is slowly decreased during the deposition process thus reducing the time required. The disadvantages are as outlined above.

At the conclusion of the deposition process, the device is returned to its upright position and the microscope slide 114 is removed from its holder 112 for further staining, other processing and evaluation as desired. The residual fluid from which the cells have been deposited is contained in the sampling compartment 232 and may be discarded with the cap assembly. The residual bulk cell suspension remains in the vial 122 and may be used or disposed of as desired. Microscope slides prepared in the described manner are suitable for use in standard laboratory procedures such as Pap staining and evaluation or immunochemical staining.

A cell transfer device is designed to transfer a representative sub-sample of a cell suspension. If too many cells are transferred, the resultant slide can be difficult to read. If too few cells are transferred, there is a danger of not having an accurate representation of the original cell suspension. The Bethesda criteria can be used in determining what constitutes an adequate sub-sample.

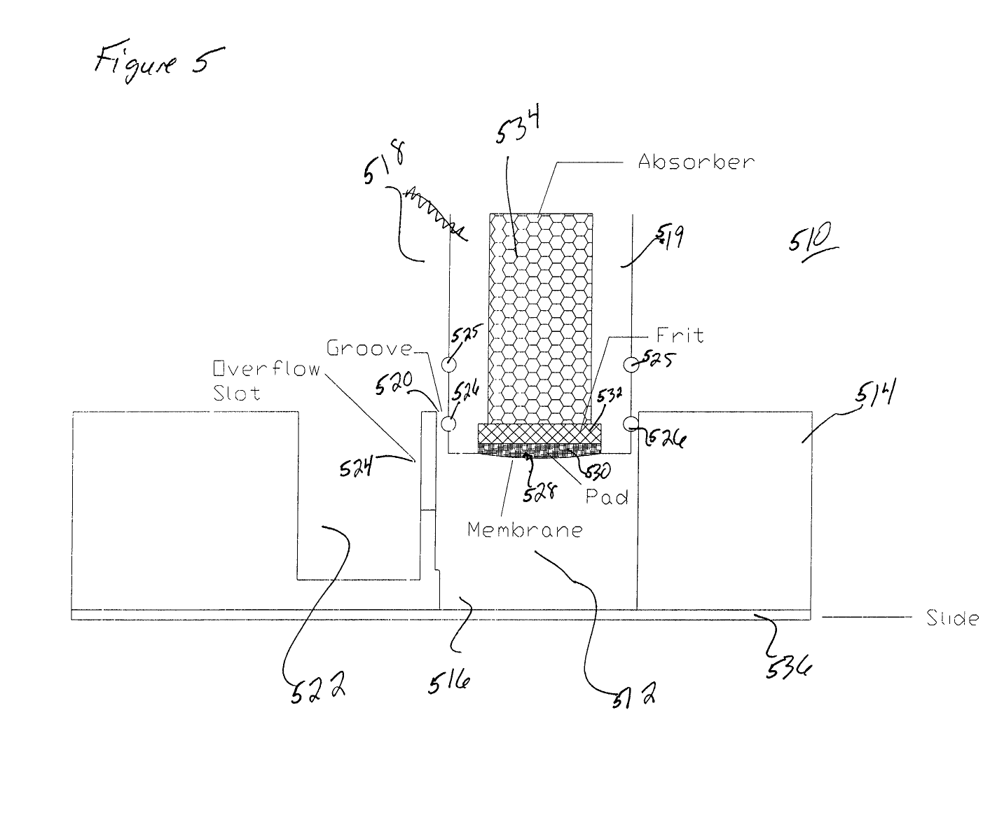

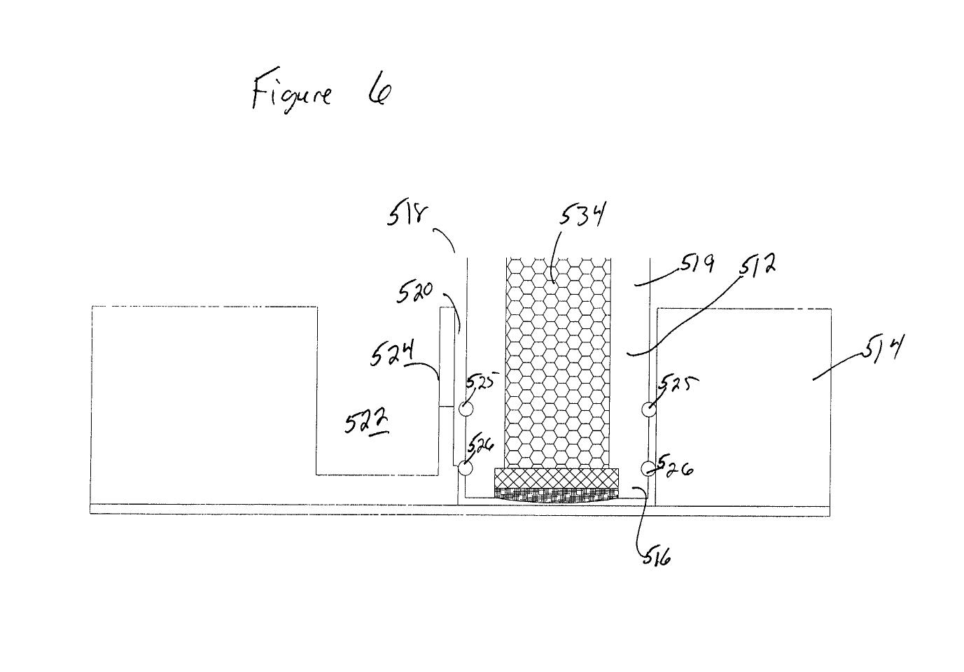

The device illustrated in FIGS. 5 and 6 performs the necessary sub-sampling of the bulk cell suspension by capturing a portion of the cells present in the suspension onto the surface of a membrane filter and subsequently transferring these captured cells to the surface of a microscope slide by direct physical contact.

This embodiment is configured as a plunger 518 that moves slideably within a compartment 512 which in turn communicates with a second compartment 522 by way of a slot or hole 524. The compartment 512 further incorporates a recess 520 in one wall that communicates with the slot or hole 524, but which does not extend to the bottom of the chamber 512 that is formed by the microscope slide 536.

The cell suspension from which the microscope slide 536 is to be prepared may be prepared separately and transferred into the chamber 512, or it may be prepared in situ by agitation of a cell collection device in preservative fluid previously placed in the chamber 512. As was described in conjunction with the previous embodiment, dimensioning of the device is determined largely by the nature and characteristics of the specific intended application. It is desirable, but not necessary that the depth of the cell suspension in the chamber 512 before introducing the plunger 518 not reach the slot or hole 524. This restriction minimizes the amount of cell suspension required.

When the plunger 518 is initially inserted into the chamber 512, features schematically illustrated as o-rings 525, 526 form a liquid tight seal between the plunger 518 and the chamber 512 except in the area of the recess 520. Advancing the plunger 518 into the chamber 512 causes any trapped air to be vented to atmosphere through the recess 520 until the plunger 512 contacts the surface of the cell suspension. Further advancing the plunger 512 causes cell suspension to be displaced from the chamber 512, through the recess 520 and the slot 524 into the chamber 522 until the seal 526 reaches the bottom of the recess 520. When the seal 526 reaches the bottom of the recess 520, it forms a closed compartment 516 containing a volume of cell suspension. The volume of cell suspension contained in the compartment 516 are determined by the dimensions of the compartment which are in turn determined by considerations previously described.

The plunger 518 can include a body 519 that fits into the chamber 512 and forms a fluid tight seal with the walls of the chamber 512 by means of the sealing features 525, 526. The bottom end of the plunger 518 can include a membrane filter 528 such as a track etch filter having a 5 to 10 micron pore size. The membrane filter 528 can be backed by a relatively thin absorbent pad 530 which, in turn, can be supported by a porous frit or similar structure 532.

The plunger 518 can also include an absorbent material 534 that will absorb a portion of the diluent present in the cell suspension sample and thus help pull cells onto the membrane filter 528. The absorbent material 534 can be selected and sized to absorb any necessary fluids without becoming saturated as the absorbent material 534 can provide at least a portion of the driving force that ensures fluid flow through the membrane filter 528 and thus ensure that a useful amount of cells are captured on the surface of the membrane filter 528. Thus, the absorbent material 534 can be formed from any suitable blotter or absorbent material. The desired sub-sample volume of cell suspension is trapped and pressurized between the face of the plunger 518 and the chamber walls.

When the seal 526 reaches the bottom of the slot 524, the closed compartment 516 containing a predetermined volume of cell suspension is formed. Continued advancement of the plunger 518 into the compartment 516 generates hydraulic pressure that forces the trapped fluid through the filter 528 and into the absorbers 530 and 534. This fluid motion is further enhanced by the absorbent action of the absorbers. During this process, the cells trapped in the chamber 516 are captured on the surface of the filter 528. Absorption of fluid causes the absorbent pad 530 to swell thus imparting a slight dome shaped distortion to the filter 528.

At the bottom of the plunger stroke (as seen in FIG. 6), the membrane 528 makes contact with the microscope slide 536. The fluid swollen pad 530 acts as a compliant element that ensures that the membrane 528 makes good contact with the slide 536 as the plunger 518 bottoms out in its stroke, much in the manner of tampo or pad printing. The microscope slide 536 can be selected such that the trapped cells adhere to it better than they do to the membrane 528, so that the captured cells are transferred to the slide 536 and remain as a defined and nearly dry spot on the slide 536 when the slide 536 is separated from the cell transfer device 510. The resulting slide 536 can then be fixed, stained and evaluated in the conventional manner.

The cell transfer device 510 can be used to analyze virtually any cell suspension. This can include analysis of menstrual fluid, urine, sputum and various lavage samples. Preferably, the cell suspension has a viscosity that is approximately equal to that of water. If the cell suspension is substantially more viscous than this, it can be diluted to a desired viscosity level. Alternatively, various chemicals are known that can substantially reduce the viscosity of a cell suspension without significantly diluting the suspension.

A particular use of the cell transfer device 510 described herein is to collect a cervical cell sample. A sample of cervical cells and possibly other cervical cellular material is collected using a brush, spatula or similar device as is well known in the art. Examples include the personal cervical cell collector described in U.S. Ser. No. 09/603,625, and the physician's collector described in U.S. Ser. No. 60/167,831, each of which are incorporated by reference herein.

The collection device is placed in a vial of liquid base preparation fluid and the vial is sonicated or agitated to release the cells from the sampling device into the solution. In a particular embodiment, this can be achieved simply by swishing the vial by hand. Thus, a cell suspension bearing the cells of interest is obtained and a volume thereof is transferred to the cell transfer device.

Upon removal of the microscope slide 536 from the device, the deposited cells may optionally be washed with a fluid to dislodge cells that are weakly retained by cell to cell contact and then stained or otherwise processed as required by the specific application.

The difference in magnitude between cell-to-slide and cell-to-cell binding forces can be exploited by gently washing the deposited cells with fluid to remove cells that are retained by the relatively weak cell-to-cell forces while leaving the cells that are retained by the relatively strong cell-to-slide forces.

A clamshell device can be configured to accept a traditional PAP test spatula or brush. The clamshell device includes a volume of cell collection fluid that can accept cells washed from the test spatula or brush. The clamshell can include a removable cover that can be replaced with a membrane support that is configured to adhere cells that are washed into the collection fluid. The clamshell device also can include pump means to move the collection fluid back and forth along the test spatula or brush. Once the cells have been collected on the membrane, they can either be transferred to a microscope slide for analysis or alternatively, can be examined optically while still on the membrane.

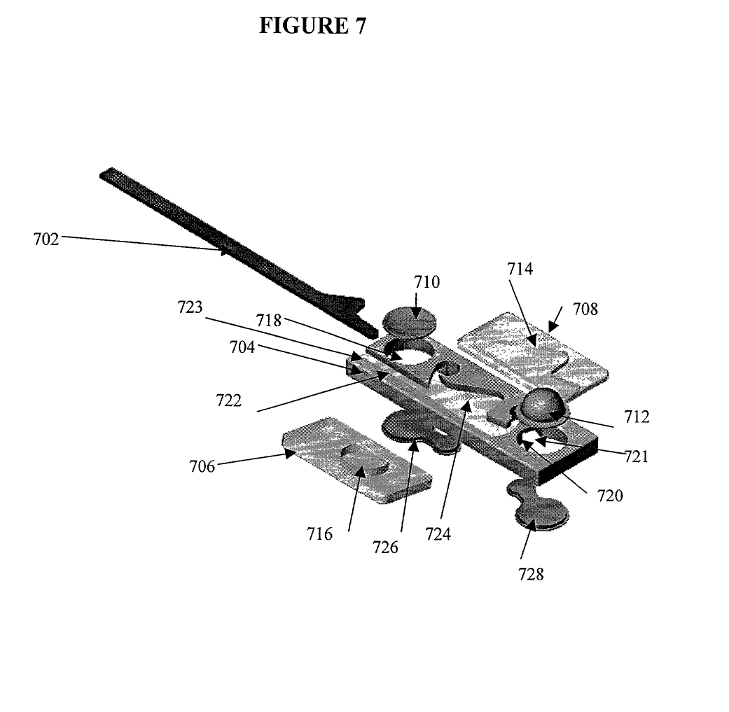

Recovery of cells from a cell collection device, preparation of a cell suspension and deposition of a monolayer of cells on a microscope slide can be integrated into a single device such as is illustrated in FIG. 7.

Specifically, FIG. 7 is an exploded perspective view of one implementation of this device. A body 704 has a recess comprised of regions 724 and 722 that are configured to accommodate the sampling portion and handle, respectively, of a cytological spatula 702. The region 722 incorporates a sealing means (not shown) that forms a fluid tight seal around the handle of the spatula when the device is in use. The regions 722 and 724 are further configured such that when the spatula 702 is in place and the lid 708 is closed, the sampling portion of the spatula is suspended within the cavity formed by the region 724 and the recess 714 in the lid 708 such that the sampling portion of the spatula is not touching the walls of the cavity. Holes 721 and 723 in the body 704 serve as ports through which fluids can be introduced into and removed from the cavity. The body can be modified to accommodate other types and designs of cell collection devices without departing from the spirit of this invention.

The hole 720 in the body 704 in conjunction with the flexible dome element 712 and the rigid bottom plate 728 form a closed reservoir that communicates with the cavity formed by the recesses 724 and 714 via the fluid port 721. A pressure actuated burst diaphragm (not shown) across the bore of port 721 isolates the reservoir (comprised of 720, 712 & 728) from the cavity prior to use. The reservoir is preferably pre-filled with a cell preservative fluid before the device is delivered to the user.

The hole 718 in conjunction with the flexible flat membrane 710 and the rigid bottom plate 726 form a closed chamber that communicates with the cavity via the fluid port 723. An optional hydrophobic vent (not shown) communicates between the chamber formed by parts 718, 710 and 726 and atmosphere.

As can be seen from FIG. 7 and the preceding description, when a cell collection device is placed in the cavity formed by the recesses 722 and 724, and the lid 708 is closed, the device consists of three chambers connected in series by fluid passages. The first chamber is filled with cell preservative fluid and is surmounted by the flexible dome 712. The second chamber contains the sampling portion of the cell collection device suspended such that the surfaces of the cell collection device are separated from the surfaces of the chamber by narrow gaps. The third chamber is filled with air and is surmounted by a flat flexible diaphragm.

The device is actuated by pressing down on the flexible dome 712. The hydrostatic pressure generated by this action bursts the diaphragm across the port 721 allowing the fluid to enter the second chamber. Due to the design of the cavity 724 and due to the cross sectional area of the gap between the sampling device and the cavity walls being smaller than those of the fluid ports 721 and 723, the fluid transits the second chamber at high velocity with considerable turbulence before exiting into the third chamber via the port 723. The fluid flow in this region is designed and intended to remove any cells present from the surface of the cell collection device and to disrupt or disaggregate the majority of cell clumps that are present.

Air displaced by this fluid flow is optionally vented via the hydrophobic vent between the third chamber atmosphere while the fluid entering the third chamber pressurizes this chamber and causes the flat flexible membrane to deform. Releasing the pressure on the dome 712 causes the fluid to be forced back through the second chamber into the first chamber due to the relaxation of the accumulated pressure and membrane deformation in the third chamber. This reverse flow provides additional cell removal and disaggregation. Several complete cycles of this process results in the first chamber containing a relatively homogeneous cell suspension. If the optional hydrophobic vent is provided, cell suspension will also be present in the second chamber. This vent is not recommended in device configurations such as illustrated in FIG. 7, but is useful in certain more complex configurations. Once the cell suspension is prepared, the lid 708 is opened and the cell collection device 702 is removed and discarded.

The device shown in FIG. 7 is configured to utilize a membrane based sub-sampling method similar to that previously described for the deposition of a monolayer of cells onto the slide. In this particular version, a second lid 706 is provided. The second lid 706 incorporates a cavity that contains a swellable absorbing material (not shown) and which is closed by a porous membrane filter (not shown) in a manner that is closely analogous to that shown for the arrangement of the membrane filter 528 and the absorber 530 in the plunger 519 of FIG. 5. Alternatively, pneumatic pressure can also be employed. When the second lid 706 is closed and pressure is applied to the dome 712, the fluid contained in the first chamber of the fluid system enters and fills the second and third chambers as previously described. However, as a portion of one wall now consists of a porous membrane filter, a certain portion of the cell suspension determined by the absorption capacity of the absorber passes through the membrane filter thereby causing those cells contained in that volume of cell suspension to be captured on the surface of the filter. The scouring action of the fluid flow parallel to the surface of the filter ensures that only a monolayer of cells is formed on the filter. In the absence of the cell sampling device, the cross sectional area of the second chamber is larger than that of the ports 721 or 723, thus limiting the flow velocity and turbulence across the surface of the filter.

Once the desired number of cells have been collected on the filter, the lid 706 is opened and a microscope slide is pressed against the filter to effect the cell transfer. The swellable absorber provides compliance to ensure good contact between the filter and the slide during this transfer. The slide is then removed from the lid 706 and processed and evaluated as desired.

Many other configurations of this device are possible. One of these configurations (not shown) retains the base 704 but replaces the lids 706 and 708 with a second body layer that incorporates fluid flow channels and a settling chamber along with provision to mount and retain a microscope slide on its upper surface. This configuration also incorporates pressure actuated diverter valves in the ports 721 and 723 that control whether the fluid flow is through the second chamber of the body 704 or into the settling chamber of the upper body. The diverter valves are designed such that if the dome 712 is pressed vigorously, fluid flow is through the body 704 in the manner previously described. This action is used to recover cells from the collection device and prepare the cell suspension. If the dome 712 is pressed gently, the diverted valves route the fluid flow into the upper body in such a manner as to fill the settling chamber with cell suspension. After the settling chamber is filled, the device is inverted and the cells are allowed to settle onto the slide as previously described. The device is then returned to its upright orientation and the slide removed for processing and evaluation.

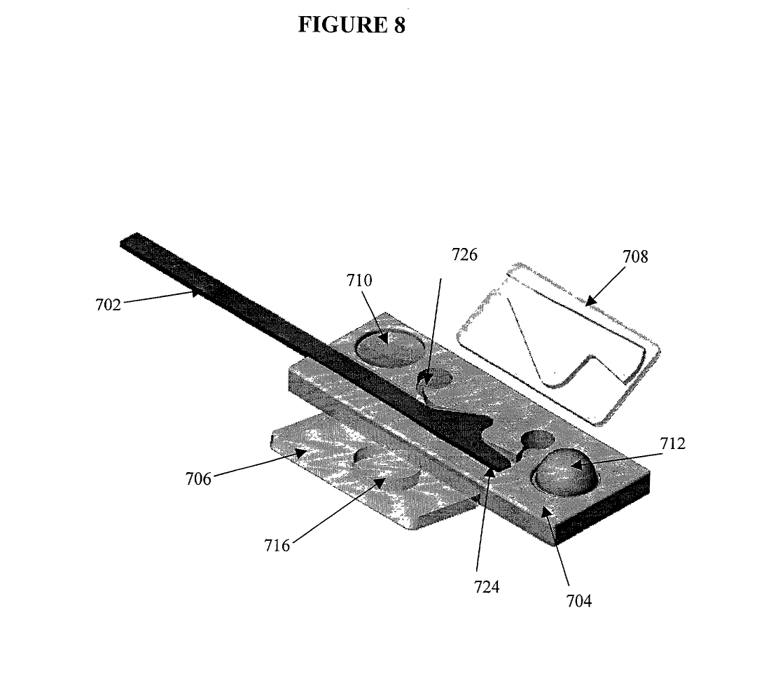

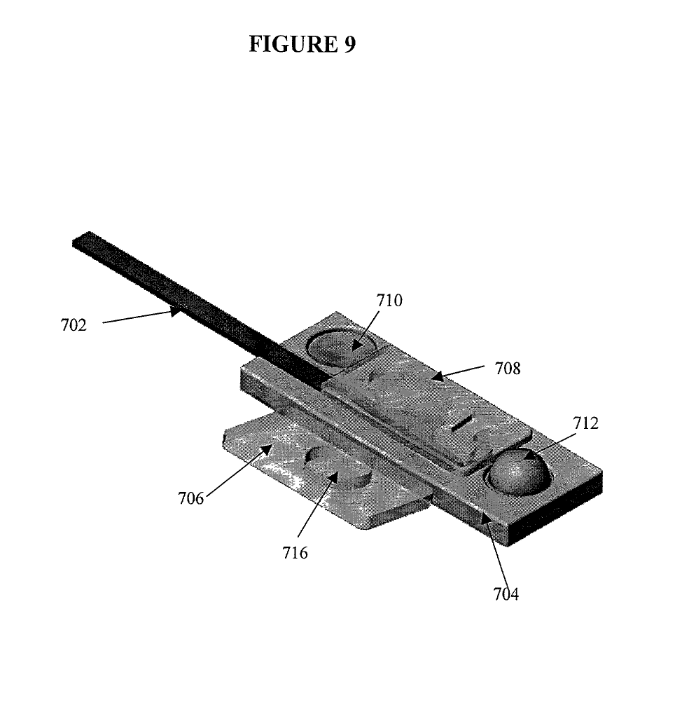

In practice, a user will preferably receive the clamshell triage device as pictured in FIG. 9, although the sampling spatula 702 does not have to be included. The user will remove cover 708 from the top of the body 704 and will insert the covers 726 and 728 if not already installed. A suitable volume of cell collection fluid is added to chambers 722 and 724 and the sampling spatula 702 (bearing cervical cells or other cells of interest) is placed as seen in FIG. 8. The cover 708 is then placed back atop the body 704 and the sampling spatula 702 as illustrated in FIG. 7.

Next, the ball pump 712 is alternatively compressed and expanded to move cell collection fluid back and forth across the sampling spatula 702, thereby washing cells from the spatula 702 into the cell collection fluid.

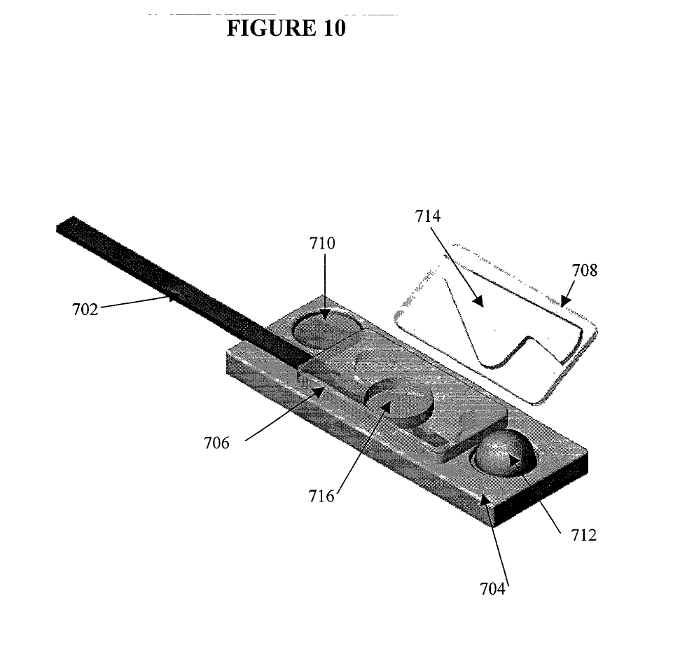

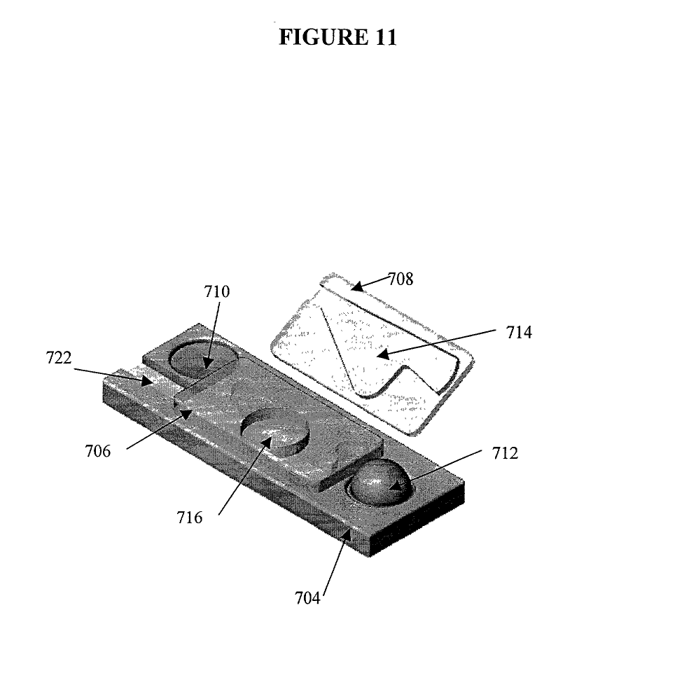

FIG. 10 illustrates the next step, which preferably includes transferring the cells from the cell collection fluid onto the collection membrane 716, which is preferably provided as part of the membrane support 706. The collection membrane 716 is placed in contact with the cell collection fluid. As shown in FIG. 11, this step can take place after withdrawing the sampling spatula 702. Once the spatula 702 is removed, the membrane 716 is in fluid contact with the cells in the cell collection fluid.

A driving force is preferred for forcing the cells into contact with the membrane 716. In one embodiment, the membrane support 706 includes an absorbent material that has sufficient absorbency to absorb the cell collection fluid that is present within the chambers 722 and 724. Thus, the cells present within the cell collection fluid are caught on the membrane 716. Once the cells are present on the membrane 716, they can be examined in any manner desired. In a preferred embodiment, they can be optically analyzed as discussed in the aforementioned 60/240,186.

While the invention has been described with reference to specific embodiments, it will be apparent to those skilled in the art that many alternatives, modifications and variations may be made. Accordingly, the present invention is intended to embrace all such alternatives, modifications and variations that may fall within the spirit and scope of the invention described herein.

We claim:

1. A device for transferring cells from a cell suspension comprising cells suspended in a fluid medium onto a substrate, comprising:
segregation means to segregate the cells in a portion of the cell suspension from the cell suspension; and
transfer means to transfer the segregated cells to a solid surface;
wherein the segregated cells are deposited on the solid surface in an approximation of a monolayer and wherein the remainder of the cell suspension is recoverable for other use.

2. The device of claim 1, wherein the transfer means permit the segregated cells to transfer to the solid surface under the influence of gravity.

3. The device of claim 1, wherein the transfer means permit the segregated cells to transfer to the solid surface under the influence of an exerted pressure.

4. The device of claim 1, wherein the transfer means permit the segregated cells to transfer to the solid surface as a result of physical contact between the transfer means and the solid surface.

5. The device of claim 1, wherein the segregation means segregate a portion of the cells in the cell suspension volumetrically.

6. The device of claim 1, further comprising cell removal means for removing excess cells, the cell removal means comprising at least one of washing, agitation and inversion.

7. The device of claim 1, further comprising absorbing means for absorbing excess fluid remaining after cell deposition.

8. The device of claim 1, wherein the solid surface comprises a microscope slide.

9. The device of claim 1, wherein the solid surface comprises a non-porous film or membrane.

10. The device of claim 1, wherein the transfer means comprises enhancing cell adhesion by applying a coating to the solid surface prior to cell deposition.

11. The device of claim 1, wherein the transfer means comprises means to capture the segregated cells on a surface of a porous medium.

12. The device of claim 1, wherein the segregation means and transfer means comprise using cell adhesion followed by one of washing, agitation and inversion.

13. The device of claim 9, wherein the segregation means are configured to permit a fluid phase of a portion of the cell suspension to pass through the porous medium, the cells contained in this portion being retained on the surface of said porous medium while the remainder of the cell suspension is displaced away from the porous medium.

14. The device of claim 13, wherein any excess fluid remaining after capture of the cells on the surface of the porous medium is captured by an absorbent material.

15. The device of claim 13, wherein the portion of the cell suspension from which cells are captured on the surface of the porous medium is determined by the volume of cell suspension that contains sufficient cells to physically block the preponderance of pores in the porous medium.

16. The device of claim 13, wherein an adsorbent material in contact with the porous medium promotes conformal and compliant contact between the porous and solid media when they are brought together.

17. The device of claim 7, wherein the absorbent material absorbs substantially all of the fluid that passes through the porous medium.

18. The device of claim 1, further comprising an end of process indicator that is configured to visually indicate when an adequate number of cells have been transferred to the solid substrate.

19. A device for depositing cells from a fluid suspension onto a solid substrate, the device comprising:
a first chamber for containing the fluid suspension, said chamber being divided into two or more contiguous zones;
a second chamber for receiving excess fluid suspension;
a channel through which excess fluid suspension can be displaced from the first chamber to the second;
retaining means of retaining the solid substrate relative to the first chamber; and
a displacement device comprising a member slideable within the first chamber, said displacement device comprising:
a body element;
a porous element; and
a fluid absorbing element.

20. The device of claim 19, wherein the displacement device is configured such that movement of the displacement device within the first zone of the first chamber traps a predetermined sub-sample of the fluid suspension within one zone of the first chamber.

21. The device of claim 19 wherein the displacement device is configured such that movement of the displacement device within the first zone of the first chamber traps a predetermined sub-sample of the fluid suspension within the second zone of the first chamber while displacing a portion of the fluid suspension from the first chamber into the second chamber.

22. The device of claim 21, wherein a volume of fluid suspension trapped with the second zone of the first chamber contains a number of cells adequate for a purpose for which the cells are being deposited.

23. The device of claim 19, wherein the displacement device is configured such that continued movement of the displacement device causes a predetermined sub-sample of the fluid suspension to pass through the porous element of the slideable element, the cells contained in the fluid suspension being captured on the surface of the porous element.

24. The device of claim 23, wherein the fluid from which the cells have been removed is absorbed by the fluid absorbing element.

25. The device of claim 23, wherein swelling of the fluid absorbing element upon fluid absorption convexly deforms the porous element and forms a compliant support for said deformed porous element.

26. The device of claim 25, wherein the deformed porous element presses upon the solid substrate thereby transferring the cells captured on the surface of the porous element from the porous element to the solid substrate.

27. The device of claim 26, wherein the compliance of the swollen absorbing element ensures uniform contact of the porous element with the solid substrate.

28. The device of claim 27, wherein continued fluid absorption by the absorbing element removes excess fluid from the cells transferred to the solid support leaving said cells nominally dry.

29. A method for depositing cells from a fluid suspension onto a solid substrate, the method comprising steps of:
moving a slideable element to trap a predetermined sub-sample of the fluid suspension in a chamber;
further moving the slideable element to transfer cells in the fluid suspension to a porous element that is attached to the slideable element with simultaneous absorption of cell-free fluid by an absorbing element; and
transferring the cells from the porous element to the solid support by pressure contact.

30. The method of claim 29, wherein the volume of trapped fluid suspension contains a number of cells that is necessary and sufficient to the intended purpose(s) for which the cells are being deposited upon the solid substrate.

31. The method of claim 29, wherein upon separation of the solid support from the device, the deposited cells are nominally dry and are in form suitable for staining or other processing.

32. A device for retrieving cells from a cell collection device, comprising:
a housing;
a first chamber within the housing;
a second chamber within the housing, the second chamber configured to accept a cell collection device, the second chamber in fluid communication with the first chamber; and
a third chamber within the housing, the third chamber in fluid communication with the first chamber and the second chamber.

33. The device of claim 32, further comprising a lid bearing a detachable membrane and an absorbent layer arranged in contact with the detachable membrane.

34. The device of claim 33, wherein the first chamber, second chamber and third chamber are configured to hold a volume of cell collection fluid and are configured to move the volume of cell collection fluid in such a way as to wash cells from the cell collection device into the fluid.

35. The device of claim 34, wherein at least a portion of the cell collection fluid is drawn into the absorbent layer, thereby capturing at least a portion of the cells on the detachable membrane.

36. The device of claim 32, further comprising a first pressure source arranged in conjunction with the first chamber and a second pressure source arranged in conjunction with the second chamber.

37. The device of claim 36, wherein the first pressure source and the second pressure source each comprise a flexible dome membrane.
